(19) United States
(12) Patent Application Publication
(10) Pub. No.: US 2015/0110729 A1
(43) Pub. Date: Apr. 23, 2015

(54) PRODUCT FOR TEMPORARILY SHAPING KERATIN FIBERS ON THE BASIS OF A COMBINATION OF SPECIFIC FILM-FORMING POLYMERS

(71) Applicant: Henkel AG & Co..KGaA, Dusseldorf (DE)

(72) Inventors: Diane Metten, Hamburg (DE); Bernd Richters, Hamburg (DE); Rene Scheffler, Ellerau (DE)

(21) Appl. No.: 14/366,473

(22) PCT Filed: Nov. 9, 2012
(86) PCT No.: PCT/EP2012/072282

§ 371 Date: Jun. 18, 2014

(30) Foreign Application Priority Data
Dec. 20, 2011 (DE) 10 2011 089 168.4

(51) Int. Cl. A61K 8/81; A61Q 5/06

(52) U.S. Cl. CPC A61K 8/81; A61Q 5/06
USPC 424/70.15

(57) ABSTRACT

Cosmetic agents, containing in a cosmetically acceptable carrier a) at least one copolymer A of 2-acrylamido-2-methylpropanesulfonic acid with one or more monomers selected from acrylic acid and methacrylic acid, b) at least one polymeric quaternary ammonium compound from the group of vinylpyrrolidone copolymers B, are particularly suitable for temporarily deforming keratinic fibers.

### CROSS REFERENCE TO RELATED APPLICATIONS

The present disclosure is a U.S. National Stage entry under 35 U.S.C. §371 based on International Application No. PCT/EP2012/072282, filed Nov. 9, 2012 which was published under PCT Article 21(2) and which claims priority to German Patent Application No. DE 10 2011 089 168.4 filed on Dec. 20, 2011, which is incorporated herein by reference in its entirety.

### TECHNICAL FIELD

The technical field is cosmetic agents based on a specific polymer combination, use of these cosmetic agents for temporarily deforming keratinic fibers, and cosmetic methods using these agents.

### BACKGROUND

Polymers are widely used in the most varied cosmetic agents. They are to be found in agents for treating skin as well as in agents for treating hair, in agents which are washed off or out again directly after use, i.e. “rinse-off products”, and in agents which remain on the skin or hair, i.e. “leave-on agents”. The polymers are used for the most varied reasons and specific properties of the polymers are exploited in each case. In agents for treating skin, in shampoos, hair rinses and hair masks, the emphasis often lies on the thickening or conditioning properties of the polymers. In agents for temporarily deforming keratinic fibers, hereinafter also known as styling agents, alongside these properties film-forming and/or setting effects are particularly desired. Polymers often also serve as auxiliaries for improving or indeed enabling deposition and fixing of other active substances and ingredients on the skin or hair. By adding suitable polymers to hair coloring agents, for example, rubbing fastness and coloring durability may be increased.

Cosmetic agents generally contain individual polymers which are specifically tailored to achieving a very specific effect. If various effects are to be achieved, a plurality of polymers must be added. However, using too many different polymers may be associated with a series of disadvantages. Problems may accordingly arise during formulation, for instance because the polymers react with one another or with other components of the agent resulting in precipitation or decomposition phenomena. Certain polymers also have a tendency to be deposited so permanently on the skin and in particular on the hair that they are no longer completely removed with normal washing and the polymer accumulates undesirably so ultimately leading to contamination of the skin or hair.

There is therefore a constant need for polymers or suitable combinations of small numbers of polymers which simultaneously exhibit as many as possible of the desired properties.

For example, in the case of styling agents, the polymers used need to give the treated hair the strongest possible hold. In addition to a high degree of hold, styling agents must meet a whole series of further requirements. These may be broadly divided into properties on the hair, properties of the respective formulation, for example properties of the foam, the gel or the sprayed aerosol, and properties which affect the handling of the styling agent, wherein properties on the hair are of particular importance. Particular mention should be made of moisture resistance, low tackiness and a well-balanced conditioning effect. Moreover, a styling agent should as far as possible be universally applicable for all hair types. If the styling agent is a gel or a paste, the polymers should additionally have thickening properties.

Accordingly, it is desirable to provide further suitable polymer combinations which are distinguished by good film-forming and/or setting properties, have a very high level of hold without having to sacrifice flexibility and good moisture resistance, in particular perspiration and water resistance, and are additionally suitable for producing stably viscous and stably transparent cosmetic compositions.

### SUMMARY

Cosmetic agents and methods of using them are provided. A cosmetic agent includes at least one copolymer A of 2-acrylamido-2-methylpropanesulfonic acid with one or more monomers selected from acrylic acid and methacrylic acid. The cosmetic agent also includes at least one polymeric quaternary ammonium compound from a group of vinylpyrrolidone copolymers B. The cosmetic agent further includes a cosmetically acceptable carrier.

A method of temporarily deforming keratinic fibers is also provided. The method includes using a cosmetic agent to temporarily deform the keratinic fibers, where the cosmetic agent includes at least one copolymer A of 2-acrylamido-2-methylpropanesulfonic acid with one or more monomers selected from acrylic acid and methacrylic acid. The cosmetic agent also includes at least one polymeric quaternary ammonium compound from a group of vinylpyrrolidone copolymers B. The cosmetic agent further includes a cosmetically acceptable carrier.

### DETAILED DESCRIPTION

An exemplary embodiment firstly provides a cosmetic agent, containing in a cosmetically acceptable carrier
a) at least one copolymer A of 2-acrylamido-2-methylpropanesulfonic acid with one or more monomers selected from acrylic acid and methacrylic acid,
b) at least one polymeric quaternary ammonium compound from the group of vinylpyrrolidone copolymers B.

The agents contain the active substances in a cosmetic carrier. For the purposes of this description, said cosmetic carrier is aqueous, alcoholic or aqueous-alcoholic. For the purposes of this description, aqueous-alcoholic carriers should be taken to be hydrous compositions containing about 3 to about 70 weight percent (wt. %) of a C₁-C₄ alcohol, in particular ethanol or isopropanol, relative to the total weight of the mixture for use. For the purposes of this description, an aqueous carrier contains at least about 30 wt. %, in particular at least about 50 wt. % water, relative to the total weight of the mixture for use. Preferred cosmetic agents contain, relative to the total weight thereof, about 40 to about 99 wt. %, preferably about 50 to about 98 wt. %, more preferably about 60 to about 95 wt. % and in particular about 70 to about 90 wt. % water. The pH value (10% solution, 20° C.) of preferred cosmetic agents amounts to about 4 to about 9, preferably about 5 to about 8 and in particular about 6 to about 7.

The cosmetic agents in an exemplary embodiment contain as first essential component at least one copolymer A of 2-acrylamido-2-methylpropanesulfonic acid (AMPS) with one or more monomers selected from acrylic acid and methacrylic acid.

Preferred agents contain at least one AMPS copolymer, selected from
a1) copolymers of acrylic acid with 2-acrylamido-2-methylpropanesulfonic acid
a2) copolymers of methacrylic acid with 2-acrylamido-2-methylpropanesulfonic acid
a3) copolymers of acrylic acid with methacrylic acid and 2-acrylamido-2-methylpropane-sulfonic acid
a4) copolymers of acrylic acid esters with 2-acrylamido-2-methylpropanesulfonic acid
a5) copolymers of methacrylic acid esters with 2-acrylamido-2-methylpropanesulfonic acid.

It has proven particularly advantageous for the cosmetic action of agents to use methacrylic acid/2-acrylamido-2-methylpropanesulfonic acid copolymers.

These can be described by the general formula

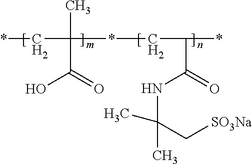

wherein the indices m and n vary depending on the molar mass of the polymer and are not intended to mean that the copolymers are block copolymers. Instead, structural units may be randomly distributed in the molecule.

Particularly preferred agents are characterized in that they contain as copolymer A copolymers of methacrylic acid with 2-acrylamido-2-methylpropanesulfonic acid with a molar mass of about 100 to about 2500 kilodaltons (kDa), preferably of about 250 to about 2000 kDa, more preferably of about 500 to about 1750 kDa and in particular of about 800 to about 1500 kDa.

Copolymers of methacrylic acid and 2-acrylamido-2-methylpropanesulfonic acid are obtainable for example under the trade name Fixomer® A-30 (Nalco). Cosmetic agents in which copolymer A is selected from the group of copolymers of methacrylic acid with 2-acrylamido-2-methylpropanesulfonic acid are therefore more preferred. In particular, those copolymers A which are based in a proportion of at least about 70 wt. %, preferably of at least about 80 wt. %, preferably of at least about 90 wt. % and in particular of at least about 95 wt. % on acrylic acid, methacrylic acid and 2-acrylamido-2-methylpropanesulfonic acid are more preferred. In particular, those copolymers A which are based in a proportion of at least about 70 wt. %, preferably of at least about 80 wt. %, preferably of at least about 90 wt. % and in particular of at least about 95 wt. % on methacrylic acid and 2-acrylamido-2-methylpropanesulfonic acid are particularly preferred.

The proportion by weight of copolymer A in the total weight of cosmetic agents preferably amounts to about 0.05 to about 10 wt. %, preferably about 0.1 to about 7.0 wt. % and in particular about 0.2 to about 5.0 wt. %.

The agents contain as second essential component a polymeric quaternary ammonium compound from the group of vinylpyrrolidone copolymers B.

Suitable copolymers B are for example
quaternized vinylpyrrolidone/dimethylaminoethyl methacrylate copolymer (INCI name Polyquaternium-11), for example the copolymers with the trade names Gafquat® 755 N and Gafquat® 734 (Gaf Co., USA) and Luviquat PQ 11 PN (BASF);
vinylpyrrolidone/imidazolimine methochloride copolymer (INCI name Polyquaternium-16), for example the copolymer Luviquat® MQ 552 (BASF);
vinylpyrrolidone/methacrylamidopropyltrimethylammonium chloride copolymer (INCI name Polyquaternium-28), for example the copolymer Gafquat® HS 100 (ISP);
methylvinylimidazolium chloride/vinylpyrrolidone copolymer (INCI name Polyquaternium-44), for example Luviquat® Ultra Care (BASF); quaternized vinylpyrrolidone/vinylcaprolactam/vinylimidazole copolymer (INCI name Polyquaternium-46), for example Luviquat® Hold (BASF); quaternized vinylpyrrolidone/vinylcaprolactam/vinylimidazole/quaternized vinylimidazole copolymer (INCI name Polyquaternium-68), for example Luviquat® Supreme (BASF).

It is more preferable for the agents to contain at least one copolymer B, selected from
b1) copolymers of vinylpyrrolidone with methacrylamidopropyltrimethylammonium chloride (MAPTAC) and/or
b2) copolymers of vinylpyrrolidone with dimethylaminoethyl methacrylate and/or
b3) copolymers of vinylpyrrolidone with dimethylaminopropylmethacrylamide and alkyldimethylpropylmethacrylamidoammonium salts.

Preferred cosmetic agents are, for example, accordingly those which contain as copolymer B copolymers of vinylpyrrolidone with methacrylamido-propyltrimethylammonium chloride (MAPTAC) (b1).

These can be described by the general formula

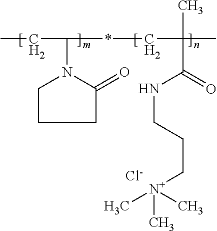

wherein the indices m and n vary depending on the molar mass of the polymer and are not intended to mean that the copolymers are block copolymers. Instead, structural units may be randomly distributed in the molecule.

More preferable cosmetic agents are characterized in that they contain as cationic polymer b1 copolymers of methacrylamidopropyltrimethylammonium chloride (MAPTAC) with vinylpyrrolidone, which contain about 40 to about 95 mol. %, preferably about 42.5 to about 90 mol. %, more preferably about 45 to about 85 mol. % and in particular about 50 to about 80 mol. % vinylpyrrolidone.

More preferred cosmetic agents are further characterized in that the copolymers b1 have molar masses of about 10 to about 1000 kDa, preferably of about 25 to about 900 kDa, more preferably of about 50 to about 800 kDa and in particular of about 100 to about 750 kDa.

A particularly preferred copolymer b1 is known according to INCI nomenclature as Polyquaternium-28. Such a polymer is obtainable for example under the trade name Gafquat® HS-100 (ISP).

In addition to the polymer(s) b1 or instead thereof, the cosmetic agents may also contain polymers b2 from the group of copolymers of vinylpyrrolidone with dimethylaminoethyl methacrylate.

These can be described by the general formula

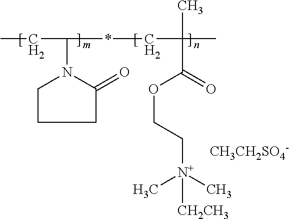

wherein the indices m and n vary depending on the molar mass of the polymer and are not intended to mean that the copolymers are block copolymers. Instead, structural units may be randomly distributed in the molecule.

More preferred cosmetic agents are characterized in that they contain as cationic polymer b2 copolymers of vinylpyrrolidone with dimethylaminoethyl methacrylate, which contain about 40 to about 95 mol. %, preferably about 42.5 to about 90 mol. %, more preferably about 45 to about 85 mol. % and in particular about 50 to about 80 mol. % vinylpyrrolidone.

More preferred cosmetic agents are further characterized in that the copolymers b2 have molar masses of about 100 to about 2500 kDa, preferably of about 250 to about 2000 kDa, more preferably of about 500 to about 1750 kDa and in particular of about 800 to about 1500 kDa.

A particularly preferred copolymer b2 is known according to INCI nomenclature as Polyquaternium-11. Such a polymer is obtainable for example under the trade name Gafquat® 755 N (ISP).

In addition to the polymer(s) b1 and/or the polymer(s) b2 or instead thereof, the cosmetic agents may also contain polymers b3 from the group of copolymers of vinylpyrrolidone with dimethylaminopropylmethacrylamide and alkyldimethylpropylmethacrylamidoammonium salts.

These can be described by the general formula

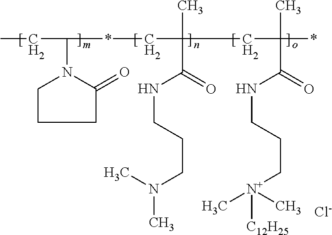

wherein the indices m, n and o vary depending on the molar mass of the polymer and are not intended to mean that the copolymers are block copolymers. Instead, structural units may be randomly distributed in the molecule.

More preferred cosmetic agents are characterized in that they contain as cationic polymer b3 copolymers of vinylpyrrolidone with dimethylaminopropylmethacrylamide and lauryldimethylpropylmethacrylamidoammonium salts.

More preferred cosmetic agents are further characterized in that they contain as cationic polymer b3 copolymers of vinylpyrrolidone with dimethylaminopropylmethacrylamide and alkyldimethylpropylmethacrylamidoammonium salts, which contain about 40 to about 95 mol. %, preferably about 42.5 to about 90 mol. %, more preferably about 45 to about 85 mol. % and in particular about 50 to about 80 mol. % vinylpyrrolidone.

Particularly preferred cosmetic agents are furthermore characterized in that the copolymers b3 have molar masses of about 10 to about 1000 kDa, preferably of about 25 to about 900 kDa, more preferably of about 50 to about 800 kDa and in particular of about 100 to about 750 kDa

A particularly preferred copolymer b3 is known according to INCI nomenclature as Polyquaternium-55. Such a polymer is obtainable for example under the trade name Styleze® W20 (ISP).

It has proven particularly advantageous for the cosmetic action of agents to use vinylpyrrolidone/methacrylamidopropyltrimethylammonium chloride copolymers. Cosmetic agents in which the copolymer B is selected from the group of copolymers of vinylpyrrolidone with methacrylamidopropyltrimethylammonium chloride are therefore more preferred. In particular, those copolymers B which are based in a proportion of at least about 70 wt. %, preferably of at least about 80 wt. %, preferably of at least about 90 wt. % and in particular of at least about 95 wt. % on vinylpyrrolidone and methacryl-amidopropyltrimethylammonium chloride are more preferred.

The proportion by weight of copolymer B in the total weight of cosmetic agents preferably amounts to about 0.05 to about 10 wt. %, preferably about 0.1 to about 7.0 wt. % and in particular about 0.2 to about 5.0 wt. %.

The agents are distinguished from cosmetic agents with alternative vinylpyrrolidone copolymers not only by the above-stated advantages but in particular also by an improved level of hold. For the cosmetic properties of the agents, a weight ratio of polymers A and B in the cosmetic agent of between about 8:1 and about 1:8, preferably between about 6:1 and about 1:6 and in particular between about 4:1 and about 1:4 has proven particularly advantageous.

Copolymer A is used in the cosmetic agents preferably in partially neutralized or neutralized form. At least one alkanolamine is preferably used for neutralization. The alkanolamines usable as an alkalizing agent are preferably selected from primary amines with a C₂-C₆ alkyl parent substance which bears at least one hydroxyl group. Particularly preferred alkanolamines are selected from the group formed by 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Alkanolamines which are particularly preferred are selected from the group 2-aminoethan-1-ol, 2-amino-2-methylpropan-1-ol and 2-amino-2-methylpropane-1,3-diol. 2-Amino-2-methylpropanol has proven to be a particularly suitable neutralizing agent. Cosmetic agents which are preferred contain at least one alkanolamine, preferably 2-amino-2-methylpropanol. 2-Amino-2-methylpropanol is used in the agents preferably in a quantity which does not exceed the quantity needed to neutralize copolymer A.

The quantities of 2-amino-2-methylpropanol used in the agents preferably amount to about 80 to 100%, more preferably about 90 to 100% and in particular about 95 to 100% of the quantity required for complete neutralization of copolymer A. In a preferred embodiment, the proportion by weight of 2-amino-2-methylpropanol in the total weight of the cosmetic agent amounts to about 0.1 to about 4.0 wt. %, preferably about 0.2 to about 3.0 wt. % and in particular about 0.5 to about 2.0 wt. %.

In addition to the previously described copolymers and carrier substances, the cosmetic agents may contain further ingredients. The group of these further ingredients in particular includes cosmetically active auxiliary substances and additives.

The cosmetic agents contain as preferred component at least one quaternary ammonium compound. Monomeric or polymeric active substances may be used as the quaternary ammonium compound.

From the plurality of possible monomeric quaternary ammonium compounds, the compounds from the groups:
trimethylalkylammonium halides;
ester quats
quaternary imidazolines
   have proven particularly effective.

The group of trimethylalkylammonium halides in particular includes the compounds of formula (Tkat1).

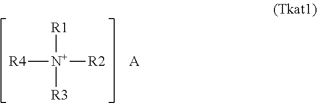

In the formula (Tkat1), R1, R2, R3 and R4 in each case mutually independently denote hydrogen, a methyl group, a phenyl group, a benzyl group, a saturated, branched or unbranched alkyl residue with a chain length of about 8 to about 30 carbon atoms, which may optionally be substituted with one or more hydroxyl groups. A denotes a physiologically acceptable anion, for example halides such as chloride or bromide and methosulfates. Examples of compounds of formula (Tkat1) are lauryltrimethylammonium chloride, cetyltrimethylammonium chloride, cetyltrimethylammonium bromide, cetyltrimethylammonium methosulfate, dicetyldimethylammonium chloride, tricetylmethylammonium chloride, stearyltrimethylammonium chloride, distearyldimethylammonium chloride, lauryldimethylbenzylammonium chloride, behenyltrimethylammonium chloride, behenyltrimethylammonium bromide, behenyltrimethylammonium methosulfate. Preferred cosmetic agents contain a monomeric quaternary ammonium compound from the group of trimethylalkylammonium halides.

Further quaternary ammonium compounds which are more preferred are the cationic betaine esters of formula (Tkat1-2.1).

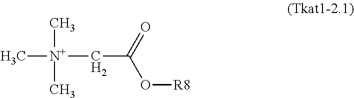

More preferred ester quats are those with the trade names Armocare VGH-70, and Dehyquart® F-75, Dehyquart® L80, Stepantex® VS 90 and Akypoquat® 131.

A further group are quaternary imidazoline compounds. The formula (Tkat2) illustrated below shows the structure of these compounds.

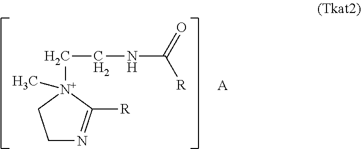

The residues R mutually independently in each case denote a saturated or unsaturated, linear or branched hydrocarbon residue with a chain length of about 8 to about 30 carbon atoms. The preferred compounds of the formula (Tkat2) in each case contain the identical hydrocarbon residue for R. The chain length of the residues R preferably amounts to about 12 to about 21 carbon atoms. A denotes an anion as previously described. Examples which are particularly are obtainable for example under NCI names Quaternium-27, Quaternium-72, Quaternium-83, Quaternium-87 and Quaternium-91. Quaternium-91 is most preferred.

With regard to cosmetic action, advantageous cosmetic agents have proven to be those in which the proportion by weight of the monomeric quaternary ammonium compound in the total weight of the agent amounts to about 0.05 to about 3.0 wt. %, preferably about 0.1 to about 2.0 wt. % and in particular about 0.2 to about 1.0 wt. %.

Suitable auxiliary substances and additives which may be mentioned are in particular additional conditioning substances.

The agent may for example contain at least one protein hydrolysate and/or one of the derivatives thereof as a conditioning substance of another compound class. Protein hydrolysates are product mixtures which are obtained by acidically, basically or enzymatically catalyzed degradation of proteins. In this description, the term “protein hydrolysates” also covers total hydrolysates and individual amino acids and the derivatives thereof and mixtures of different amino acids. The molecular weight of the protein hydrolysates which may be used is between about 75, the molecular weight of glycine, and about 200,000, the molecular weight preferably amounting to about 75 to about 50,000 and particularly preferably to about 75 to about 20,000 daltons.

The agent may furthermore contain at least one vitamin, a provitamin, a vitamin precursor and/or one of the derivatives thereof as a conditioning substance. Preferred vitamins, provitamins and vitamin precursors are those which are conventionally assigned to groups A, B, C, E, F and H.

Like the addition of glycerol and/or propylene glycol, the addition of panthenol increases the flexibility of the polymer film formed using the agent.

The agents may furthermore contain at least one plant extract, but also mono- or oligosaccharides and/or lipids as conditioning substance.

Oil bodies are furthermore suitable as a conditioning substance. Natural and synthetic cosmetic oil bodies include, for example, vegetable oils, liquid paraffin oils, isoparaffin oils and synthetic hydrocarbons and di-n-alkyl ethers having a total of between about 12 and about 36 carbon (C) atoms, in particular about 12 to about 24 C atoms. Preferred cosmetic agents contain at least one oil body, preferably at least one oil body from the group of silicone oils. The group of silicone oils in particular includes dimethicones, which also include cyclomethicones, amino-functional silicones and dimethiconols. The dimethicones may be both linear and branched and cyclic or cyclic and branched. Suitable silicone oils or silicone gums are in particular dialkyl- and alkylarylsiloxanes, such as for example dimethylpolysiloxane and methylphenylpolysiloxane, and the alkoxylated, quaternized or also anionic derivatives thereof Preference is given to cyclic and linear polydialkylsiloxanes, the alkoxylated and/or aminated derivatives thereof, dihydroxypoly-dimethylsiloxanes and polyphenylalkylsiloxanes.

Ester oils, i.e. esters of C₆-C₃₀ fatty acids with C₂-C₃₀ fatty alcohols, preferably monoesters of fatty acids with alcohols having 2 to about 24 C atoms such as for example isopropyl myristate (Rilanit® IPM), isononanoic acid C₁₆₋₁₈ alkyl esters (Cetiol® SN), 2-ethylhexyl palmitate (Cegesoft® 24), stearic acid 2-ethylhexyl ester (Cetiol® 868), cetyl oleate, glycerol tricaprylate, coco fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are further preferred conditioning oil bodies.

Additional suitable conditioning substances are dicarboxylic acid esters, symmetrical, asymmetrical or cyclic esters of carbonic acid with fatty alcohols, trifatty acid esters of saturated and/or unsaturated linear and/or branched fatty acids with glycerol, or fatty acid partial glycerides, which should be understood to mean monoglycerides, diglycerides and the technical mixtures thereof

With regard to cosmetic action, advantageous cosmetic agents have proven to be those in which the proportion by weight of the oil body in the total weight of the agent amounts to about 0.01 to about 5.0 wt. %, preferably about 0.02 to about 4.0 wt. % and in particular about 0.05 to about 2.0 wt. %.

The following tables show the composition of some preferred cosmetic agents (details in wt. % relative to the total weight of the cosmetic agent unless otherwise stated).

| | Formula 1 | | Formula 2 | | Formula 3 | | Formula 4 | Formula 5 |
|---|---|---|---|---|---|---|---|---|
| Copolymer A | 0.05 to | 10 | 0.1 to | 7.0 | 0.2 to | 5.0 | 0.4 | 1.2 |
| Copolymer B | 0.05 to | 10 | 0.1 to | 7.0 | 0.2 to | 5.0 | 0.8 | 0.8 |
| Water, misc. | ad 100 | | ad 100 | | ad 100 | | ad 100 | ad 100 |
| | Formula 6 | | Formula 7 | | Formula 8 | | Formula 9 | Formula 10 |
| Copolymer A¹⁾ | 0.05 to | 10 | 0.1 to | 7.0 | 0.2 to | 5.0 | 0.4 | 1.2 |
| Copolymer B | 0.05 to | 10 | 0.1 to | 7.0 | 0.2 to | 5.0 | 0.8 | 0.8 |
| Water, misc. | ad 100 | | ad 100 | | ad 100 | | ad 100 | ad 100 |
| | Formula 11 | | Formula 12 | | Formula 13 | | Formula 14 | Formula 15 |
| Copolymer A | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| Water, misc. | ad 100 | | ad 100 | | ad 100 | | ad 100 | ad 100 |
| | Formula 16 | | Formula 17 | | Formula 18 | | Formula 19 | Formula 20 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| Water, misc. | ad 100 | | ad 100 | | ad 100 | | ad 100 | ad 100 |
| | Formula 21 | | Formula 22 | | Formula 23 | | Formula 24 | Formula 25 |
| Copolymer A | 0.05 to | 10 | 0.1 to | 7.0 | 0.2 to | 5.0 | 0.4 | 1.2 |
| Copolymer B | 0.05 to | 10 | 0.1 to | 7.0 | 0.2 to | 5.0 | 0.8 | 0.8 |
| Alkanolamine³⁾ | 80 to | 100 | 90 to | 100 | 95 to | 100 | 98 | 98 |
| Water, misc. | ad 100 | | ad 100 | | ad 100 | | ad 100 | ad 100 |
| | Formula 26 | | Formula 27 | | Formula 28 | | Formula 29 | Formula 30 |
| Copolymer A¹⁾ | 0.05 to | 10 | 0.1 to | 7.0 | 0.2 to | 5.0 | 0.4 | 1.2 |
| Copolymer B | 0.05 to | 10 | 0.1 to | 7.0 | 0.2 to | 5.0 | 0.8 | 0.8 |
| Alkanolamine³⁾ | 80 to | 100 | 90 to | 100 | 95 to | 100 | 98 | 98 |
| Water, misc. | ad 100 | | ad 100 | | ad 100 | | ad 100 | ad 100 |
| | Formula 31 | | Formula 32 | | Formula 33 | | Formula 34 | Formula 35 |
| Copolymer A | 0.05 to | 10 | 0.1 to | 7.0 | 0.2 to | 5.0 | 0.4 | 1.2 |
| Copolymer B | 0.05 to | 10 | 0.1 to | 7.0 | 0.2 to | 5.0 | 0.8 | 0.8 |
| Alkanolamine³⁾ | 80 to | 100 | 90 to | 100 | 95 to | 100 | 98 | 98 |
| Water, misc. | ad 100 | | ad 100 | | ad 100 | | ad 100 | ad 100 |
| | Formula 36 | | Formula 37 | | Formula 38 | | Formula 39 | Formula 40 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| Alkanolamine³⁾ | 80 to | 100 | 90 to | 100 | 95 to | 100 | 98 | 98 |
| Water, misc. | ad 100 | | ad 100 | | ad 100 | | ad 100 | ad 100 |
| | Formula 41 | | Formula 42 | | Formula 43 | | Formula 44 | Formula 45 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| 2-Amino-2-methylpropanol³⁾ | 80 to | 100 | 90 to | 100 | 95 to | 100 | 98 | 98 |
| Water, misc. | ad 100 | | ad 100 | | ad 100 | | ad 100 | ad 100 |
| | Formula 46 | | Formula 47 | | Formula 48 | | Formula 49 | Formula 50 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| Alkyltrimethylammonium | 0.05 to | 3.0 | 0.1 to | 2.0 | 0.2 to | 1.0 | 0.5 | 0.3 |
| chloride | | | | | | | | |
| Water, misc. | ad 100 | | ad 100 | | ad 100 | | ad 100 | ad 100 |
| | Formula 51 | | Formula 52 | | Formula 53 | | Formula 54 | Formula 55 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| Oil bodies | 0.01 to | 5.0 | 0.02 to | 4.0 | 0.05 to | 2.0 | 0.1 | 0.1 |
| Water, misc. | ad 100 | | ad 100 | | ad 100 | | ad 100 | ad 100 |
| | Formula 56 | | Formula 57 | | Formula 58 | | Formula 59 | Formula 60 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| Water | 40 to | 99 | 50 to | 98 | 60 to | 95 | 97 | 92 |
| Misc. | ad 100 | | ad 100 | | ad 100 | | ad 100 | ad 100 |
| | Formula 61 | | Formula 62 | | Formula 63 | | Formula 64 | Formula 65 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| Alkyltrimethylammonium | 0.05 to | 3.0 | 0.1 to | 2.0 | 0.2 to | 1.0 | 0.5 | 0.3 |
| chloride | | | | | | | | |
| Water | 40 to 99 | | 50 to 98 | | 60 to 95 | | 97 | 92 |
| Misc. | ad 100 | | ad 100 | | ad 100 | | ad 100 | ad 100 |
| | Formula 66 | | Formula 67 | | Formula 68 | | Formula 69 | Formula 70 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| Oil bodies | 0.01 to | 5.0 | 0.02 to | 4.0 | 0.05 to | 2.0 | 0.1 | 0.1 |
| Water | 40 to | 99 | 50 to | 98 | 60 to | 95 | 97 | 92 |
| Misc. | ad 100 | | ad 100 | | ad 100 | | ad 100 | ad 100 |
| | Formula 71 | | Formula 72 | | Formula 73 | | Formula 74 | Formula 75 |
| Copolymer A¹⁾ | 0.05 to | 10 | 0.1 to | 7.0 | 0.2 to | 5.0 | 0.4 | 1.2 |
| Copolymer B²⁾ | 0.05 to | 10 | 0.1 to | 7.0 | 0.2 to | 5.0 | 0.8 | 0.8 |
| 2-Amino-2-methylpropanol³⁾ | 80 to | 100 | 90 to | 100 | 95 to | 100 | 98 | 98 |
| Oil bodies | 0.01 to | 5.0 | 0.02 to | 4.0 | 0.05 to | 2.0 | 0.1 | 0.1 |
| Water | 40 to | 99 | 50 to | 98 | 60 to | 95 | 97 | 92 |

| | | | | | |
|---|---|---|---|---|---|
| Misc. | ad 100 | ad 100 | ad 100 | ad 100 | ad 100 |

¹⁾Copolymer A, which is based in a proportion of at least about 95 wt. % on methacrylic acid and 2-acrylamido-2-methylpropanesulfonic acid.
²⁾Copolymer B, which is based in a proportion of at least about 95 wt. % on the monomers vinylpyrrolidone and methacrylamidopropyltrimethylammonium chloride
³⁾% of the quantity required for complete neutralization of copolymer A

The cosmetic agents may be formulated in any forms conventional for cosmetic agents, for example in the form of solutions, which may for example be applied onto the hair as a hair lotion, in the form of creams, emulsions, waxes, gels or also surfactant-containing foaming solutions or other preparations which are suitable for use on the hair. In an alternative embodiment, these agents may however also assume gel or cream form, wherein transparent gels are more preferred.

The compositions are in particular highly suitable for stabilizing gas bubbles in the agent. In this way, air or other gases or gas mixtures can be readily incorporated into the agents in such a manner as to be stable in the long-term. This may optionally proceed during production of the agents, by exposing the agent to gas, preferably air, before packaging and packaging a product containing visible gas bubbles. The agents preferably assume the form of a foam. A foam is here a preparation comprising gas filled bubbles surrounded by liquid (liquid foam) or solid (stiff foam) walls. The compositions listed in the following table are preferably stiff foams. The density of preferred compositions amounts to about 0.3 to about 1.0 grams per cubic centimeter (g/cm³), preferably about 0.4 to about 0.9 g/cm³ and in particular about 0.5 to about 0.8 g/cm³. Such foams may for example be produced by beating the preparation in a suitable mixer or by exposure to a suitable gas, preferably air.

The following tables show the composition of some preferred cosmetic foams. In this table the left-hand column (“formula x”) refers in each case to one of the exemplary cosmetic compositions listed in the tables disclosed further above. The further columns two to seven (“density”) in each case indicate the density of the corresponding cosmetic composition.

In other words, a cosmetic preparation according to line 12, column 5 of the following table comprises a cosmetic agent according to formula 11 with a density of 0.44 g/cm³.

| | Density [g/cm³] | | | | | |
|---|---|---|---|---|---|---|
| Formula 1 | 0.3 to 1.0 | 0.93 | 0.4 to 0.9 | 0.44 | 0.5 to 0.8 | 0.61 |
| Formula 2 | 0.3 to 1.0 | 0.93 | 0.4 to 0.9 | 0.44 | 0.5 to 0.8 | 0.61 |
| Formula 3 | 0.3 to 1.0 | 0.93 | 0.4 to 0.9 | 0.44 | 0.5 to 0.8 | 0.61 |
| Formula 4 | 0.3 to 1.0 | 0.93 | 0.4 to 0.9 | 0.44 | 0.5 to 0.8 | 0.61 |
| Formula 5 | 0.3 to 1.0 | 0.93 | 0.4 to 0.9 | 0.44 | 0.5 to 0.8 | 0.61 |
| Formula 6 | 0.3 to 1.0 | 0.93 | 0.4 to 0.9 | 0.44 | 0.5 to 0.8 | 0.61 |
| Formula 7 | 0.3 to 1.0 | 0.93 | 0.4 to 0.9 | 0.44 | 0.5 to 0.8 | 0.61 |
| Formula 8 | 0.3 to 1.0 | 0.93 | 0.4 to 0.9 | 0.44 | 0.5 to 0.8 | 0.61 |
| Formula 9 | 0.3 to 1.0 | 0.93 | 0.4 to 0.9 | 0.44 | 0.5 to 0.8 | 0.61 |
| Formula 10 | 0.3 to 1.0 | 0.93 | 0.4 to 0.9 | 0.44 | 0.5 to 0.8 | 0.61 |
| Formula 11 | 0.3 to 1.0 | 0.93 | 0.4 to 0.9 | 0.44 | 0.5 to 0.8 | 0.61 |
| Formula 12 | 0.3 to 1.0 | 0.93 | 0.4 to 0.9 | 0.44 | 0.5 to 0.8 | 0.61 |
| Formula 13 | 0.3 to 1.0 | 0.93 | 0.4 to 0.9 | 0.44 | 0.5 to 0.8 | 0.61 |
| Formula 14 | 0.3 to 1.0 | 0.93 | 0.4 to 0.9 | 0.44 | 0.5 to 0.8 | 0.61 |
| Formula 15 | 0.3 to 1.0 | 0.93 | 0.4 to 0.9 | 0.44 | 0.5 to 0.8 | 0.61 |
| Formula 16 | 0.3 to 1.0 | 0.93 | 0.4 to 0.9 | 0.44 | 0.5 to 0.8 | 0.61 |
| Formula 17 | 0.3 to 1.0 | 0.93 | 0.4 to 0.9 | 0.44 | 0.5 to 0.8 | 0.61 |
| Formula 18 | 0.3 to 1.0 | 0.93 | 0.4 to 0.9 | 0.44 | 0.5 to 0.8 | 0.61 |
| Formula 19 | 0.3 to 1.0 | 0.93 | 0.4 to 0.9 | 0.44 | 0.5 to 0.8 | 0.61 |
| Formula 20 | 0.3 to 1.0 | 0.93 | 0.4 to 0.9 | 0.44 | 0.5 to 0.8 | 0.61 |
| Formula 21 | 0.3 to 1.0 | 0.93 | 0.4 to 0.9 | 0.44 | 0.5 to 0.8 | 0.61 |
| Formula 22 | 0.3 to 1.0 | 0.93 | 0.4 to 0.9 | 0.44 | 0.5 to 0.8 | 0.61 |
| Formula 23 | 0.3 to 1.0 | 0.93 | 0.4 to 0.9 | 0.44 | 0.5 to 0.8 | 0.61 |
| Formula 24 | 0.3 to 1.0 | 0.93 | 0.4 to 0.9 | 0.44 | 0.5 to 0.8 | 0.61 |
| Formula 25 | 0.3 to 1.0 | 0.93 | 0.4 to 0.9 | 0.44 | 0.5 to 0.8 | 0.61 |
| Formula 26 | 0.3 to 1.0 | 0.93 | 0.4 to 0.9 | 0.44 | 0.5 to 0.8 | 0.61 |
| Formula 27 | 0.3 to 1.0 | 0.93 | 0.4 to 0.9 | 0.44 | 0.5 to 0.8 | 0.61 |
| Formula 28 | 0.3 to 1.0 | 0.93 | 0.4 to 0.9 | 0.44 | 0.5 to 0.8 | 0.61 |
| Formula 29 | 0.3 to 1.0 | 0.93 | 0.4 to 0.9 | 0.44 | 0.5 to 0.8 | 0.61 |
| Formula 30 | 0.3 to 1.0 | 0.93 | 0.4 to 0.9 | 0.44 | 0.5 to 0.8 | 0.61 |
| Formula 31 | 0.3 to 1.0 | 0.93 | 0.4 to 0.9 | 0.44 | 0.5 to 0.8 | 0.61 |
| Formula 32 | 0.3 to 1.0 | 0.93 | 0.4 to 0.9 | 0.44 | 0.5 to 0.8 | 0.61 |
| Formula 33 | 0.3 to 1.0 | 0.93 | 0.4 to 0.9 | 0.44 | 0.5 to 0.8 | 0.61 |
| Formula 34 | 0.3 to 1.0 | 0.93 | 0.4 to 0.9 | 0.44 | 0.5 to 0.8 | 0.61 |
| Formula 35 | 0.3 to 1.0 | 0.93 | 0.4 to 0.9 | 0.44 | 0.5 to 0.8 | 0.61 |
| Formula 36 | 0.3 to 1.0 | 0.93 | 0.4 to 0.9 | 0.44 | 0.5 to 0.8 | 0.61 |
| Formula 37 | 0.3 to 1.0 | 0.93 | 0.4 to 0.9 | 0.44 | 0.5 to 0.8 | 0.61 |
| Formula 38 | 0.3 to 1.0 | 0.93 | 0.4 to 0.9 | 0.44 | 0.5 to 0.8 | 0.61 |
| Formula 39 | 0.3 to 1.0 | 0.93 | 0.4 to 0.9 | 0.44 | 0.5 to 0.8 | 0.61 |
| Formula 40 | 0.3 to 1.0 | 0.93 | 0.4 to 0.9 | 0.44 | 0.5 to 0.8 | 0.61 |
| Formula 41 | 0.3 to 1.0 | 0.93 | 0.4 to 0.9 | 0.44 | 0.5 to 0.8 | 0.61 |
| Formula 42 | 0.3 to 1.0 | 0.93 | 0.4 to 0.9 | 0.44 | 0.5 to 0.8 | 0.61 |
| Formula 43 | 0.3 to 1.0 | 0.93 | 0.4 to 0.9 | 0.44 | 0.5 to 0.8 | 0.61 |
| Formula 44 | 0.3 to 1.0 | 0.93 | 0.4 to 0.9 | 0.44 | 0.5 to 0.8 | 0.61 |
| Formula 45 | 0.3 to 1.0 | 0.93 | 0.4 to 0.9 | 0.44 | 0.5 to 0.8 | 0.61 |
| Formula 46 | 0.3 to 1.0 | 0.93 | 0.4 to 0.9 | 0.44 | 0.5 to 0.8 | 0.61 |
| Formula 47 | 0.3 to 1.0 | 0.93 | 0.4 to 0.9 | 0.44 | 0.5 to 0.8 | 0.61 |
| Formula 48 | 0.3 to 1.0 | 0.93 | 0.4 to 0.9 | 0.44 | 0.5 to 0.8 | 0.61 |
| Formula 49 | 0.3 to 1.0 | 0.93 | 0.4 to 0.9 | 0.44 | 0.5 to 0.8 | 0.61 |
| Formula 50 | 0.3 to 1.0 | 0.93 | 0.4 to 0.9 | 0.44 | 0.5 to 0.8 | 0.61 |
| Formula 51 | 0.3 to 1.0 | 0.93 | 0.4 to 0.9 | 0.44 | 0.5 to 0.8 | 0.61 |
| Formula 52 | 0.3 to 1.0 | 0.93 | 0.4 to 0.9 | 0.44 | 0.5 to 0.8 | 0.61 |
| Formula 53 | 0.3 to 1.0 | 0.93 | 0.4 to 0.9 | 0.44 | 0.5 to 0.8 | 0.61 |
| Formula 54 | 0.3 to 1.0 | 0.93 | 0.4 to 0.9 | 0.44 | 0.5 to 0.8 | 0.61 |
| Formula 55 | 0.3 to 1.0 | 0.93 | 0.4 to 0.9 | 0.44 | 0.5 to 0.8 | 0.61 |
| Formula 56 | 0.3 to 1.0 | 0.93 | 0.4 to 0.9 | 0.44 | 0.5 to 0.8 | 0.61 |
| Formula 57 | 0.3 to 1.0 | 0.93 | 0.4 to 0.9 | 0.44 | 0.5 to 0.8 | 0.61 |
| Formula 58 | 0.3 to 1.0 | 0.93 | 0.4 to 0.9 | 0.44 | 0.5 to 0.8 | 0.61 |
| Formula 59 | 0.3 to 1.0 | 0.93 | 0.4 to 0.9 | 0.44 | 0.5 to 0.8 | 0.61 |
| Formula 60 | 0.3 to 1.0 | 0.93 | 0.4 to 0.9 | 0.44 | 0.5 to 0.8 | 0.61 |
| Formula 61 | 0.3 to 1.0 | 0.93 | 0.4 to 0.9 | 0.44 | 0.5 to 0.8 | 0.61 |
| Formula 62 | 0.3 to 1.0 | 0.93 | 0.4 to 0.9 | 0.44 | 0.5 to 0.8 | 0.61 |
| Formula 63 | 0.3 to 1.0 | 0.93 | 0.4 to 0.9 | 0.44 | 0.5 to 0.8 | 0.61 |
| Formula 64 | 0.3 to 1.0 | 0.93 | 0.4 to 0.9 | 0.44 | 0.5 to 0.8 | 0.61 |
| Formula 65 | 0.3 to 1.0 | 0.93 | 0.4 to 0.9 | 0.44 | 0.5 to 0.8 | 0.61 |
| Formula 66 | 0.3 to 1.0 | 0.93 | 0.4 to 0.9 | 0.44 | 0.5 to 0.8 | 0.61 |
| Formula 67 | 0.3 to 1.0 | 0.93 | 0.4 to 0.9 | 0.44 | 0.5 to 0.8 | 0.61 |
| Formula 68 | 0.3 to 1.0 | 0.93 | 0.4 to 0.9 | 0.44 | 0.5 to 0.8 | 0.61 |
| Formula 69 | 0.3 to 1.0 | 0.93 | 0.4 to 0.9 | 0.44 | 0.5 to 0.8 | 0.61 |
| Formula 70 | 0.3 to 1.0 | 0.93 | 0.4 to 0.9 | 0.44 | 0.5 to 0.8 | 0.61 |
| Formula 71 | 0.3 to 1.0 | 0.93 | 0.4 to 0.9 | 0.44 | 0.5 to 0.8 | 0.61 |
| Formula 72 | 0.3 to 1.0 | 0.93 | 0.4 to 0.9 | 0.44 | 0.5 to 0.8 | 0.61 |
| Formula 73 | 0.3 to 1.0 | 0.93 | 0.4 to 0.9 | 0.44 | 0.5 to 0.8 | 0.61 |
| Formula 74 | 0.3 to 1.0 | 0.93 | 0.4 to 0.9 | 0.44 | 0.5 to 0.8 | 0.61 |
| Formula 75 | 0.3 to 1.0 | 0.93 | 0.4 to 0.9 | 0.44 | 0.5 to 0.8 | 0.61 |

In an alternative embodiment, the cosmetic agents may be packaged as pump or aerosol sprays. In addition to further active and auxiliary substances, preferred aerosol sprays contain a propellant. Propellants (propellant gases) suitable are propane, n-butane, iso-butane, dimethyl ether (DME), nitrogen, air, nitrous oxide, 1,1-difluoroethane, specifically both individually and in combination. Hydrophilic propellant gases, such as for example carbon dioxide, may also advantageously be used for the purposes of the present invention if a small proportion of hydrophilic gases is selected and a lipophilic propellant gas (for example propane/butane) is present in excess. Dimethyl ether, propane, n-butane, iso-butane and mixtures of these propellant gases are more preferred. The use of propane/butane mixtures or isobutane is particularly preferred. Cosmetic agents which, relative to the total weight thereof, contain the propellant in a quantity of about 2.0-20 wt. %, preferably about 4.0-15 wt. % and more preferably about 5.0-10 wt. %, are preferred.

The following tables show the composition of some preferred propellant-containing cosmetic agents. In this table the left-hand column (“formula x”) refers in each case to one of the exemplary cosmetic compositions listed in the tables disclosed further above. The further columns two to seven (“propellant”) in each case indicate the quantity of propellant added to the corresponding cosmetic composition. These indications in “wt. %” relate to the total weight of the cosmetic composition of the respective “formula x” without propellant.

In other words, a cosmetic preparation according to line 12, column 5 of the following table comprises a 20:1 mixture of the propellant-free cosmetic agent according to formula 11 with a propane/butane mixture.

| | Propellant [wt. %] | | | | | |
|---|---|---|---|---|---|---|
| Formula 1 | 2.0 to 10 | 5.0 | 2.0 to 10 P/B* | 5.0 P/B | 2.0 to 10 iB** | 5.0 iB |
| Formula 2 | 2.0 to 10 | 5.0 | 2.0 to 10 P/B* | 5.0 P/B | 2.0 to 10 iB** | 5.0 iB |
| Formula 3 | 2.0 to 10 | 5.0 | 2.0 to 10 P/B* | 5.0 P/B | 2.0 to 10 iB** | 5.0 iB |
| Formula 4 | 2.0 to 10 | 5.0 | 2.0 to 10 P/B* | 5.0 P/B | 2.0 to 10 iB** | 5.0 iB |
| Formula 5 | 2.0 to 10 | 5.0 | 2.0 to 10 P/B* | 5.0 P/B | 2.0 to 10 iB** | 5.0 iB |
| Formula 6 | 2.0 to 10 | 5.0 | 2.0 to 10 P/B* | 5.0 P/B | 2.0 to 10 iB** | 5.0 iB |
| Formula 7 | 2.0 to 10 | 5.0 | 2.0 to 10 P/B* | 5.0 P/B | 2.0 to 10 iB** | 5.0 iB |
| Formula 8 | 2.0 to 10 | 5.0 | 2.0 to 10 P/B* | 5.0 P/B | 2.0 to 10 iB** | 5.0 iB |
| Formula 9 | 2.0 to 10 | 5.0 | 2.0 to 10 P/B* | 5.0 P/B | 2.0 to 10 iB** | 5.0 iB |
| Formula 10 | 2.0 to 10 | 5.0 | 2.0 to 10 P/B* | 5.0 P/B | 2.0 to 10 iB** | 5.0 iB |
| Formula 11 | 2.0 to 10 | 5.0 | 2.0 to 10 P/B* | 5.0 P/B | 2.0 to 10 iB** | 5.0 iB |
| Formula 12 | 2.0 to 10 | 5.0 | 2.0 to 10 P/B* | 5.0 P/B | 2.0 to 10 iB** | 5.0 iB |
| Formula 13 | 2.0 to 10 | 5.0 | 2.0 to 10 P/B* | 5.0 P/B | 2.0 to 10 iB** | 5.0 iB |
| Formula 14 | 2.0 to 10 | 5.0 | 2.0 to 10 P/B* | 5.0 P/B | 2.0 to 10 iB** | 5.0 iB |
| Formula 15 | 2.0 to 10 | 5.0 | 2.0 to 10 P/B* | 5.0 P/B | 2.0 to 10 iB** | 5.0 iB |
| Formula 16 | 2.0 to 10 | 5.0 | 2.0 to 10 P/B* | 5.0 P/B | 2.0 to 10 iB** | 5.0 iB |
| Formula 17 | 2.0 to 10 | 5.0 | 2.0 to 10 P/B* | 5.0 P/B | 2.0 to 10 iB** | 5.0 iB |
| Formula 18 | 2.0 to 10 | 5.0 | 2.0 to 10 P/B* | 5.0 P/B | 2.0 to 10 iB** | 5.0 iB |
| Formula 19 | 2.0 to 10 | 5.0 | 2.0 to 10 P/B* | 5.0 P/B | 2.0 to 10 iB** | 5.0 iB |
| Formula 20 | 2.0 to 10 | 5.0 | 2.0 to 10 P/B* | 5.0 P/B | 2.0 to 10 iB** | 5.0 iB |
| Formula 21 | 2.0 to 10 | 5.0 | 2.0 to 10 P/B* | 5.0 P/B | 2.0 to 10 iB** | 5.0 iB |
| Formula 22 | 2.0 to 10 | 5.0 | 2.0 to 10 P/B* | 5.0 P/B | 2.0 to 10 iB** | 5.0 iB |
| Formula 23 | 2.0 to 10 | 5.0 | 2.0 to 10 P/B* | 5.0 P/B | 2.0 to 10 iB** | 5.0 iB |
| Formula 24 | 2.0 to 10 | 5.0 | 2.0 to 10 P/B* | 5.0 P/B | 2.0 to 10 iB** | 5.0 iB |
| Formula 25 | 2.0 to 10 | 5.0 | 2.0 to 10 P/B* | 5.0 P/B | 2.0 to 10 iB** | 5.0 iB |
| Formula 26 | 2.0 to 10 | 5.0 | 2.0 to 10 P/B* | 5.0 P/B | 2.0 to 10 iB** | 5.0 iB |
| Formula 27 | 2.0 to 10 | 5.0 | 2.0 to 10 P/B* | 5.0 P/B | 2.0 to 10 iB** | 5.0 iB |
| Formula 28 | 2.0 to 10 | 5.0 | 2.0 to 10 P/B* | 5.0 P/B | 2.0 to 10 iB** | 5.0 iB |
| Formula 29 | 2.0 to 10 | 5.0 | 2.0 to 10 P/B* | 5.0 P/B | 2.0 to 10 iB** | 5.0 iB |
| Formula 30 | 2.0 to 10 | 5.0 | 2.0 to 10 P/B* | 5.0 P/B | 2.0 to 10 iB** | 5.0 iB |
| Formula 31 | 2.0 to 10 | 5.0 | 2.0 to 10 P/B* | 5.0 P/B | 2.0 to 10 iB** | 5.0 iB |
| Formula 32 | 2.0 to 10 | 5.0 | 2.0 to 10 P/B* | 5.0 P/B | 2.0 to 10 iB** | 5.0 iB |
| Formula 33 | 2.0 to 10 | 5.0 | 2.0 to 10 P/B* | 5.0 P/B | 2.0 to 10 iB** | 5.0 iB |
| Formula 34 | 2.0 to 10 | 5.0 | 2.0 to 10 P/B* | 5.0 P/B | 2.0 to 10 iB** | 5.0 iB |
| Formula 35 | 2.0 to 10 | 5.0 | 2.0 to 10 P/B* | 5.0 P/B | 2.0 to 10 iB** | 5.0 iB |
| Formula 36 | 2.0 to 10 | 5.0 | 2.0 to 10 P/B* | 5.0 P/B | 2.0 to 10 iB** | 5.0 iB |
| Formula 37 | 2.0 to 10 | 5.0 | 2.0 to 10 P/B* | 5.0 P/B | 2.0 to 10 iB** | 5.0 iB |
| Formula 38 | 2.0 to 10 | 5.0 | 2.0 to 10 P/B* | 5.0 P/B | 2.0 to 10 iB** | 5.0 iB |
| Formula 39 | 2.0 to 10 | 5.0 | 2.0 to 10 P/B* | 5.0 P/B | 2.0 to 10 iB** | 5.0 iB |
| Formula 40 | 2.0 to 10 | 5.0 | 2.0 to 10 P/B* | 5.0 P/B | 2.0 to 10 iB** | 5.0 iB |
| Formula 41 | 2.0 to 10 | 5.0 | 2.0 to 10 P/B* | 5.0 P/B | 2.0 to 10 iB** | 5.0 iB |
| Formula 42 | 2.0 to 10 | 5.0 | 2.0 to 10 P/B* | 5.0 P/B | 2.0 to 10 iB** | 5.0 iB |
| Formula 43 | 2.0 to 10 | 5.0 | 2.0 to 10 P/B* | 5.0 P/B | 2.0 to 10 iB** | 5.0 iB |
| Formula 44 | 2.0 to 10 | 5.0 | 2.0 to 10 P/B* | 5.0 P/B | 2.0 to 10 iB** | 5.0 iB |
| Formula 45 | 2.0 to 10 | 5.0 | 2.0 to 10 P/B* | 5.0 P/B | 2.0 to 10 iB** | 5.0 iB |
| Formula 46 | 2.0 to 10 | 5.0 | 2.0 to 10 P/B* | 5.0 P/B | 2.0 to 10 iB** | 5.0 iB |
| Formula 47 | 2.0 to 10 | 5.0 | 2.0 to 10 P/B* | 5.0 P/B | 2.0 to 10 iB** | 5.0 iB |
| Formula 48 | 2.0 to 10 | 5.0 | 2.0 to 10 P/B* | 5.0 P/B | 2.0 to 10 iB** | 5.0 iB |
| Formula 49 | 2.0 to 10 | 5.0 | 2.0 to 10 P/B* | 5.0 P/B | 2.0 to 10 iB** | 5.0 iB |
| Formula 50 | 2.0 to 10 | 5.0 | 2.0 to 10 P/B* | 5.0 P/B | 2.0 to 10 iB** | 5.0 iB |
| Formula 51 | 2.0 to 10 | 5.0 | 2.0 to 10 P/B* | 5.0 P/B | 2.0 to 10 iB** | 5.0 iB |
| Formula 52 | 2.0 to 10 | 5.0 | 2.0 to 10 P/B* | 5.0 P/B | 2.0 to 10 iB** | 5.0 iB |
| Formula 53 | 2.0 to 10 | 5.0 | 2.0 to 10 P/B* | 5.0 P/B | 2.0 to 10 iB** | 5.0 iB |
| Formula 54 | 2.0 to 10 | 5.0 | 2.0 to 10 P/B* | 5.0 P/B | 2.0 to 10 iB** | 5.0 iB |
| Formula 55 | 2.0 to 10 | 5.0 | 2.0 to 10 P/B* | 5.0 P/B | 2.0 to 10 iB** | 5.0 iB |
| Formula 56 | 2.0 to 10 | 5.0 | 2.0 to 10 P/B* | 5.0 P/B | 2.0 to 10 iB** | 5.0 iB |
| Formula 57 | 2.0 to 10 | 5.0 | 2.0 to 10 P/B* | 5.0 P/B | 2.0 to 10 iB** | 5.0 iB |
| Formula 58 | 2.0 to 10 | 5.0 | 2.0 to 10 P/B* | 5.0 P/B | 2.0 to 10 iB** | 5.0 iB |
| Formula 59 | 2.0 to 10 | 5.0 | 2.0 to 10 P/B* | 5.0 P/B | 2.0 to 10 iB** | 5.0 iB |
| Formula 60 | 2.0 to 10 | 5.0 | 2.0 to 10 P/B* | 5.0 P/B | 2.0 to 10 iB** | 5.0 iB |
| Formula 61 | 2.0 to 10 | 5.0 | 2.0 to 10 P/B* | 5.0 P/B | 2.0 to 10 iB** | 5.0 iB |
| Formula 62 | 2.0 to 10 | 5.0 | 2.0 to 10 P/B* | 5.0 P/B | 2.0 to 10 iB** | 5.0 iB |
| Formula 63 | 2.0 to 10 | 5.0 | 2.0 to 10 P/B* | 5.0 P/B | 2.0 to 10 iB** | 5.0 iB |
| Formula 64 | 2.0 to 10 | 5.0 | 2.0 to 10 P/B* | 5.0 P/B | 2.0 to 10 iB** | 5.0 iB |
| Formula 65 | 2.0 to 10 | 5.0 | 2.0 to 10 P/B* | 5.0 P/B | 2.0 to 10 iB** | 5.0 iB |
| Formula 66 | 2.0 to 10 | 5.0 | 2.0 to 10 P/B* | 5.0 P/B | 2.0 to 10 iB** | 5.0 iB |
| Formula 67 | 2.0 to 10 | 5.0 | 2.0 to 10 P/B* | 5.0 P/B | 2.0 to 10 iB** | 5.0 iB |
| Formula 68 | 2.0 to 10 | 5.0 | 2.0 to 10 P/B* | 5.0 P/B | 2.0 to 10 iB** | 5.0 iB |
| Formula 69 | 2.0 to 10 | 5.0 | 2.0 to 10 P/B* | 5.0 P/B | 2.0 to 10 iB** | 5.0 iB |
| Formula 70 | 2.0 to 10 | 5.0 | 2.0 to 10 P/B* | 5.0 P/B | 2.0 to 10 iB** | 5.0 iB |
| Formula 71 | 2.0 to 10 | 5.0 | 2.0 to 10 P/B* | 5.0 P/B | 2.0 to 10 iB** | 5.0 iB |
| Formula 72 | 2.0 to 10 | 5.0 | 2.0 to 10 P/B* | 5.0 P/B | 2.0 to 10 iB** | 5.0 iB |
| Formula 73 | 2.0 to 10 | 5.0 | 2.0 to 10 P/B* | 5.0 P/B | 2.0 to 10 iB** | 5.0 iB |
| Formula 74 | 2.0 to 10 | 5.0 | 2.0 to 10 P/B* | 5.0 P/B | 2.0 to 10 iB** | 5.0 iB |
| Formula 75 | 2.0 to 10 | 5.0 | 2.0 to 10 P/B* | 5.0 P/B | 2.0 to 10 iB** | 5.0 iB |

*“P/B” corresponds to a propane/butane mixture
**“iB” corresponds to isobutane

As explained above, the agents have advantageous hair-fixing properties. The present description therefore also provides a method for temporarily deforming keratinic fibers, in which a composition of an exemplary embodiment is applied onto the keratinic fibers. The present description also provides use of a cosmetic agent for temporarily deforming keratinic fibers. As explained above, the agents are distinguished in particular by improved hold in the case of temporary deformation of keratinic fibers. The present description therefore additionally provides the use of a cosmetic agent to improve hold in the case of temporary deformation of keratinic fibers.

### EXAMPLES

The humidity resistance (High Humidity Curl Retention; HHCR) of the temporary hair deformation achieved using the following three hair cosmetic agents was determined

| | Invention 1 | Comparison 1 | Comparison 2 |
|---|---|---|---|
| Fixomer A-30¹ | 6.25 | 12.5 | — |
| Gafquat HS-100² | 3.75 | — | 7.5 |
| Sodium benzoate | 0.3 | 0.3 | 0.3 |
| D-Panthenol (75%) | 0.2 | 0.2 | 0.2 |
| Dow Corning 939³ | 0.2 | 0.2 | 0.2 |
| Dehyquart A CA⁴ | 1.0 | 1.0 | 1.0 |
| Castor Oil, hydrogenated, | 0.2 | 0.2 | 0.2 |
| 40 EO | | | |
| Perfume | 0.1 | 0.1 | 0.1 |
| Water, misc. | ad 100 | ad 100 | ad 100 |

¹Copolymers of methacrylic acid and 2-acrylamido-2-methylpropanesulfonic acid (12% in water)
²Vinylpyrrolidone/methacrylamidopropyltrimethylammonium chloride copolymer (INCI: Polyquaternium-28, 20% in water)
³Silicone preparation (INCI: Amodimethicone, Trideceth-12, Cetrimonium Chloride)
⁴Trimethylhexadecylammonium chloride

To determine High Humidity Curl Retention, standardized strands of hair from Kerling (item no. 827560) of the “European Natural” hair type, color 6/0, of a length (Lₘₐₓ) of 220 mm and a weight of 0.6 g were used. The strands were washed with a 12.5 wt. % sodium laureth sulfate solution by way of preparation. The strands of hair were dried overnight in a drying oven at 318 K.

0.18 g of the compositions were applied onto a strand of hair and rubbed in. The strand was then wound onto a curler (Fripac-medis, diameter 7 mm, item no. D-1203) and dried overnight at room temperature.

The curlers were carefully removed and the strands hung up. The length of the curls were in each case measured (L₀) and the strands placed in a conditioning cabinet. They were stored there at 294 K and a relative atmospheric humidity of 85% over a period of 6 h, after which the length of the curls was remeasured (Lₜ).

Five test strands per composition were correspondingly treated and measured.

High Humidity Curl Retention (HHCR) was calculated according to the following formula and the arithmetic mean of the HHCR values for the 5 test strands was determined for each composition:

$$HHCR = \frac{{L}_{max} - {L}_{t}}{{L}_{max} - {L}_{0}}$$

| | Invention 1 | Comparison 1 | Comparison 2 |
|---|---|---|---|
| HHCR | 85% | 77% | 38% |

The measurement data reveal the synergistic action of the polymer combination in composition Invention 1.

While at least one exemplary embodiment has been presented in the foregoing detailed description, it should be appreciated that a vast number of variations exist. It should also be appreciated that the exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

1. A cosmetic agent comprising:
a) at least one copolymer A of 2-acrylamido-2-methylpropanesulfonic acid with one or more monomers selected from acrylic acid and methacrylic acid;
b) at least one polymeric quaternary ammonium compound from a group of vinylpyrrolidone copolymers B; and
a cosmetically acceptable carrier.

2. The cosmetic agent according to claim 1 wherein the at least one copolymer A is of 2-acrylamido-2-methylpropanesulfonic acid with methacrylic acid.

3. The cosmetic agent according to claim 1, characterized in that the proportion by weight of the copolymer A in the total weight of the agent amounts to about 0.05 weight percent (wt. %) to about 10 wt. %.

4. The cosmetic agent according claim 1, characterized in that the copolymer B is selected from the group of copolymers of vinylpyrrolidone with methacrylamidopropyltrimethylammonium chloride.

5. The cosmetic agent according to claim 1, characterized in that the proportion by weight of the copolymer B in the total weight of the agent amounts to about 0.05 weight percent (wt. %) to about 10 wt. %.

6. The cosmetic agent according to claim 1, characterized in that the cosmetic agent furthermore comprises at least one alkanolamine.

7. The cosmetic agent according to claim 1, characterized in that the cosmetic agent furthermore comprises at least one monomeric quaternary ammonium compound.

8. The cosmetic agent according to claim 1, characterized in that the cosmetic agent furthermore comprises at least one oil body.

9. A method of temporarily deforming keratinic fibers, the method comprising the steps of:
using a cosmetic agent to temporarily deform the keratinic fibers, wherein the cosmetic agent comprises:
a) at least one copolymer A of 2-acrylamido-2-methylpropanesulfonic acid with one or more monomers selected from acrylic acid and methacrylic acid;
b) at least one polymeric quaternary ammonium compound from a group of vinylpyrrolidone copolymers B; and
a cosmetically acceptable carrier.

10. The method of claim 9 further comprising:
applying the cosmetic agent onto the keratinic fibers.

11. The cosmetic agent according to claim 1, characterized in that the proportion by weight of the copolymer A in the total weight of the agent amounts to about 0.1 weight percent (wt. %) to about 7.0 wt %.

12. The cosmetic agent according to claim 1, characterized in that the proportion by weight of the copolymer A in the total weight of the agent amounts to about 0.2 weight percent (wt. %) to about 5.0 wt. %.

13. The cosmetic agent according to claim 1, characterized in that the proportion by weight of the copolymer B in the total weight of the agent amounts to about 0.1 weight percent (wt. %) to about 7.0 wt. %.

14. The cosmetic agent according to claim 1, characterized in that the proportion by weight of the copolymer B in the total weight of the agent amounts to about 0.2 weight percent (wt. %) to about 5.0 wt %.

15. The cosmetic agent according to claim 1, characterized in that the cosmetic agent furthermore comprises 2-amino-2-methylpropanol.

16. The cosmetic agent according to claim 1, characterized in that the cosmetic agent furthermore comprises a monomeric quaternary ammonium compound from the group of trimethylalkylammonium halides.

17. The cosmetic agent according to claim 1, characterized in that the cosmetic agent furthermore comprises at least one oil body from the group of silicone oils.
